(19) United States
(12) Patent Application Publication
(10) Pub. No.: US 2006/0094673 A1
(43) Pub. Date: May 4, 2006

(54) Polymer surfactants for gene therapy applications

(76) Inventor: Benjamin Chu, Setauket, NY (US)

Correspondence Address: PITNEY HARDIN LLP, 7 TIMES SQUARE, NEW YORK, NY, 10036-7311, US

(21) Appl. No.: 10/512,228

(22) PCT Filed: Apr. 23, 2003
(86) PCT No.: PCT/US03/12777

§ 371 Date: Dec. 27, 2005

Related U.S. Application Data
(63) Continuation-in-part of application No. 60/374,657, filed on Apr. 23, 2002.

(51) Int. Cl. A61K 48/00

(52) U.S. Cl. 514/44; 525/54.2; 525/54.1

(57) ABSTRACT

A composition and method capable of delivering pharmaceutical or biomedical materials includes a tri-block surfactant having a hydrophilic block, a charged water-soluble block and a hydrophobic block.

This invention was supported by Grant No. DMR 9984102 from the National Science Foundation and Grant No. 1HG01386-07 from the National Human Genome Research Institute, both of which may have rights in the invention.

### BACKGROUND OF THE INVENTION

This invention relates to a composition and method capable of delivering pharmaceutical or biomedical materials including a tri-block surfactant having a hydrophilic block, a charged water-soluble block and hydrophobic block. The tri-block surfactant provides a biodegradable carrier for pharmaceutical or biomedical materials.

Viral vectors have encountered serious safety concerns, such as inflammatory reactions and virus replication. In contrast, non-viral vectors, although markedly lower in inefficiency at the present time, offer flexibility in design and great potential in meeting requirements such as receptor recognition, improved safety profile, protection of DNA from degradation by nucleases and improved immunogenicity for gene therapy applications. While many attempts have been made to develop non-viral therapy gene delivery systems by using cationic liposomes or polymers and combinations thereof, the results of such efforts have not been very successful.

Cationic polymers, such as poly-L-lysine (PLL), poly (ethylenimine) (PEI), poly(dimethylaminoethylmethacrylate) (PDMAEMA), diethylaminoethyl-dextran and chitosan, have been used in complexation studies, with the positively charged PEI considered to be the most promising delivery candidate. Linear PEI is marketed commercially as a transfection agent under the trademark ExGen 500. Unfortunately, the DNA-PEI complex is only partly soluble in an aqueous environment. More importantly, PEI is quite toxic.

In the search for effective drug delivery and DNA delivery carriers, synthetic polymers and hybrids with natural polymers have been studied, including biodegradable polymers and their copolymers as well as the use of core-shell nanoparticles.

Drug and DNA delivery and release behavior is governed by the ability of the carrier to pass through lipophilic cell membranes (transfection). Bioactive agents such as proteins and DNA may lose their bioactivities when exposed to hydrophobic surfaces and to degradation products of polymers.

### SUMMARY OF THE INVENTION

It is an object of the invention to provide a non-viral carrier for pharmaceutical and biomedical materials.

Another object of the invention is to provide a nonviral carrier which can transport intact pharmaceutical and biomedical materials across cell membranes.

These and other objects of the invention are achieved by providing a surfactant comprising a triblock copolymer including a hydrophilic block, a charged water-soluble block and a hydrophobic block. The surfactant can form complexes with pharmaceutical or biomedical materials and transport these materials across cell membranes.

If the pharmaceutical or biomedical material is positively charged, the charged water-soluble block is negatively charged, while a positively charged water-soluble block will be used if the biomedical or pharmaceutical material is negatively charged.

### BRIEF DESCRIPTION OF THE DRAWINGS

FIG. 1 is an illustration of a synthesis of a linear polyethylene glycol-polylactide-polylysine tri-block copolymer surfactant in accordance with the invention;

FIG. 2 is an illustration of a synthesis of a linear polyethylene glycol-polylysine-polylactide tri-block polymer surfactant in accordance with the invention;

FIG. 3 is an illustration of a synthesis of a tri-arm block polymer surfactant in accordance with the invention;

FIG. 4(a), (b), (c) are schematic illustrations of (a) micelle formation of a tri-block polymer surfactant in a hydrophilic environment, (b) a tri-arm tri-block copolymer (c) micelle formation of a tri-block copolymer in a hydrophobic environment;

FIG. 5(a) is a schematic illustration of a complex with DNA, 5(b) is a schematic illustration of condensed DNA encapsulated by the tri-block surfactant where the condensed DNA forms a supramolecular complex with oppositely charged blocks. The surface of the complex encapsulates the DNA and the other two blocks, one hydrophilic and one hydrophobic, make the supramolecular assembly soluble in either hydrophilic or hydrophobic environment;

FIG. 6 is a schematic illustration of a transport of a DNA surfactant complex across a cell membrane; and

FIG. 7 is a schematic illustration of disassembly of the DNA-surfactant complex inside the cell.

### DETAILED DESCRIPTION OF THE PREFERRED EMBODIMENTS

Polyelectrolyte-surfactant complexes (PSC's) are unique materials with the ability to spontaneously self-assemble into highly ordered nanostructures. PSC's are formed by the complex formation of polyelectrolytes and oppositely charged ionic surfactants, usually in aqueous solution. Many biologically active materials such as DNA, polypeptides, polysaccharides, etc. are natural polyelectrolytes.

PSCs can be considered as microphase-separated materials, showing segregation into polar and hydrophobic domains on a nanometer length scale. The microphase separation can and often does lead to long-range order, so that these PSCs form ordered crystal-like structures, macro-lattices, with constants of typically a few nanometers.

In other, less-ordered PSCs, discrete microphase-separated complexes are formed, usually but not always spherical in shape. The less ordered PSCs are not arranged on a three dimensional lattice, but only show a liquid-like short range order, or when sufficiently diluted, no ordered arrangement at all. This type of PSC is referred as colloidal polyelectrolyte surfactant complexes (CPSCs).

Parameters influencing the structure and morphologies of PSCs (and CPSCs) include the chain length, charge density, crosslinker density, backbone hydrophobicity and persistence length of the polyelectrolyte; the nature of the solvent, the chemical composition of the solvent, whether the solvent is a pure fluid or mixture, the physical properties of the solvent including dielectric constant and viscosity; with respect to the surfactant, the number and type of head charges, geometry of hydrophobic tail(s), chain length, geometry of hydrophilic part, topology, i.e. single or double tail; and external parameters such as pH, ionic strength, PSC concentration, temperature, pressure, nature of counterion and stoichiometry.

In the present invention a polyelectrolyte and tri-block surfactant form a polyelectrolyte surfactant complex which can cross a cell membrane and deliver the essentially encapsulated polyelectrolyte to the cell without significant loss of the polyelectrolyte's bioactivity. Preferably the PSC is a CPSC.

The tri-block copolymer includes hydrophilic, charged and hydrophobic blocks. Preferably the blocks are FDA approved and the hydrophobic block is biodegradable. The presence of a hydrophilic block and a hydrophobic block ensures solubility of the tri-block copolymer in both the aqueous environment and hydrophobic environment. The presence of the hydrophobic block can also be used to modify the surface properties of the PSC and thereby further protect the PSC components in the supramolecular structure. The biodegradable nature of the hydrophobic block can be selected to permit degradation after the soluble supramolecular complex crosses the cell membrane.

Examples of suitable hydrophilic block components include but are not limited to polyethylene oxide, polyethylene glycol (PEG) or other water-soluble neutral polymers, such as polypropylene oxide at low temperatures.

Examples of suitable charged block components include but are not limited to poly(aminoacids), polyacrylic acid, polyethylenemine and poly(dimethylaminoethyl-methacrylate), as well as polysaccharides, including but are not limited to chitin/chitosan and hyaluronic acid. Examples of poly(aminoacids) include but are not limited to poly(L-lysine), poly(diethylamino-L-glutamine), polyarginine and polyornithine.

Examples of suitable hydrophobic components include but are limited to poly(glycolide), poly(lactide), poly(lactide-co-glycolide), polyanhydride, poly(dioxanone), sebacic acid, poly(ε-caprolactone), and polyhydroxybutyrate as well as polyalkylene oxide, e.g., polypropylene oxide at high temperatures. Polypropylene oxide is hydrophilic at low temperatures and hydrophobic at high temperatures.

Any pharmaceutical or biomedical agent which is charged can be included in the PSC. By “pharmaceutical or biomedical agent” is meant a biologically active molecule that can be used in the treatment, cure, prevention or diagnosis of disease or is otherwise used to enhance physical or mental well being in humans or other animals. The biologically active molecules include but are not limited to proteins, peptides, oligonucleotides, DNA, RNA and polysaccharides and of course any other molecules having such activity.

Proteins and peptides which may be used in accordance with the present invention include enzymes such as proteases (e.g. bromelain, papain, collagenase, elastase), lipases (e.g. phospholipase C), esterases, glucosidases, hyaluronidase, exfoliating enzymes; antibodies and antibody derived actives, such as monoclonal antibodies, polyclonal antibodies, single chain antibodies and the like; reductases; oxidases; peptide hormones; natural structural skin proteins, such as elastin, collagen, reticulin and the like; anti-oxidants such as superoxide dismutase, catalase and glutathione; free-radical scavenging proteins; DNA-repair enzymes, for example T4 endonuclease 5 and P53; antimicrobial peptides, such as magainin and cecropin; a milk protein; a silk protein or peptide; and any active fragments, derivatives of these proteins and peptides; and mixtures thereof an anti-viral agent (such as acyclovir); an anti-hemorrhoid compound, an anti-wart agent (such as podophyllotoxin) and a plant extract and mixtures thereof.

Cytokines can also be incorporated into the delivery system. The cytokines include vascular endothelial growth factor (VEGF), endothelial cell growth factor (ECGF), fibroblast growth factor (FGF), insulin-like growth factor (IGF), bone morphogenic growth factor (BMP), platelet-derived growth factor (PDGF), epidermal growth factor (EGF), thrombopoietin (TPO), interleukins (IL1-IL15), interferons (IFN), erythropoietin (EPO), ciliary neurotrophic factor (CNTF), colony stimulating factors (G-CSF, M-CSF, GM-CSF), glial cell-derived neurotrophic factor (GDNF), leukemia inhibitory factor (LIF), and macrophage inflammatory proteins (MIP-1a,-1b,-2).

Genetic material can also be incorporated in the delivery system. Gene therapy can be used to introduce an exogenous gene in an animal to supplement or replace a defective or missing gene. For example, genes including but not limited to, genes encoding for HLA-B, insulin, adenosine deaminase, cytokines and coagulant factor VIII can be incorporated into the matrix and released over a fixed time period. The desired material can be operably linked to a variety of promoters well known in the art. Examples of promoters include, but are not limited to, an endogenous adenovirus promoter, such as the E1 a promoter or the Ad2 major late promoter (MLP) or a heterologous eucaryotic promoter, for example a phosphoglycerate kinase (PGK) promoter or a cytomegalovirus (CMV) promoter. Similarly, those of ordinary skill in the art can construct adenoviral vectors using endogenous or heterologous poly A addition signals.

In an alternate embodiment the tri-block surfactant encapsulates the desired gene when it is in a condensed state by using kinetic processing under non-equilibrium conditions. The resultant supramolecular complex with a condensed DNA core has a duel amphiphilic property and is targeted specifically to overcome challenges in the solubility of those complexes under a variety of aqueous and hydrophobic solvent environments, the enhancement of gene transfection and gene protection, as well as the final gene delivery to the nucleus in the cell.

The DNA can first be collapsed into a condensed state by dissolving or suspending DNA chains in a poor or non-solvent. For example, the DNA can be condensed in a solvent mixture of 94% w/w % N,N-dimethyl formamide and 6% w/w % water. In coil-to-globule transition phenomena volume changes of the order of thousands can be observed. There will be a competition between the formation of globules or other condensed states and the aggregation of DNA molecules. Thus, kinetic processing under non-equilibrium conditions will be considered.

The condensed DNA molecule(s) can be coated with a polyelectrolyte-surfactant complex shell using the strong electrostatic interactions between the DNA and the tri-functional surfactant that contains an oppositely charged block. This complex formation involves another volume contraction that could reduce the supramolecular complex to even smaller sizes. It is noted that DNA-surfactant complexes are generally insoluble. However, each surfactant molecule is covalently attached to both a hydrophilic block and a hydrophobic block. Thus, the supramolecular complex can be designed to be soluble in both the hydrophobic environment needed for the DNA condensation, encapsulation, and cell membrane penetration and the aqueous environment needed for gene delivery and movements inside the cell.

The following examples illustrate preparation of a tri-block surfactant in accordance with the invention, the formation of complexes with DNA, transfection of cells and disassembly of the complex.

The tri-block surfactant can be linear ELB or EBL where E=hydrophilic block, L=positively or negatively charged block and B=hydrophobic block. In addition, the notation EBL implies that after biodegradation, E and L are no longer covalently linked together, while ELB implies that E and L remain covalently bonded after biodegradation. The morphology of the tri-arm star ELB indicates that after biodegradation, E and L will remain covalently linked. Preferably, the PEG used in the following examples has a molar mass of less than 20 k.

### EXAMPLE 1

### Synthesis of Biodegradable Tri-Block Copolymer PEG-b-PLA-PLL

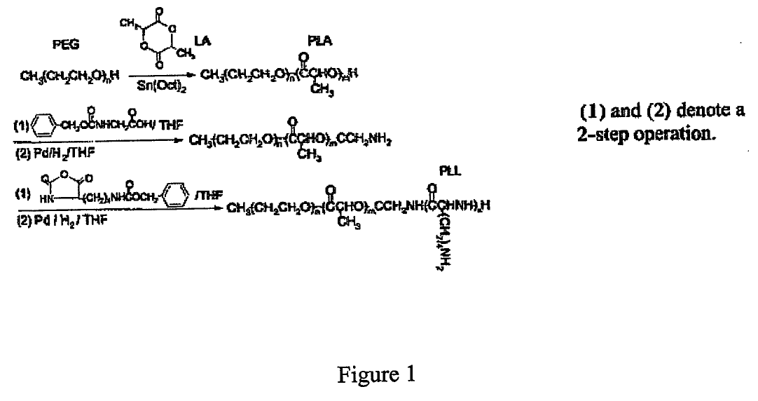

PLA-b-PEG could be synthesized by polymerization of lactide in the presence of PEG as described in Zhu et al. J. Appl. Polym. Sci., 39, 1-9 (1990). The hydroxyl end group of the diblock polymers can then be converted to an amino group by reaction with N-CBZ-glycine as set forth in C. Y. Won et al., J. Appl. Polym. Sci., 70, 953-963 (1998), and subsequent deprotection. The terminal amino group initiates the polymerization of NCA to give the tri-block copolymer in accordance with Kricheldorf, “Aminoacid-N-carboxy-anhydride and related heterocycles” Spinger-Vertag, Berlin (1987) pp. 3-58. NCA is not commercially available. This synthesis is shown in FIG. 1.

### EXAMPLE 2

### Synthesis of Tri-Block Copolymer PEG-PLL-PLA

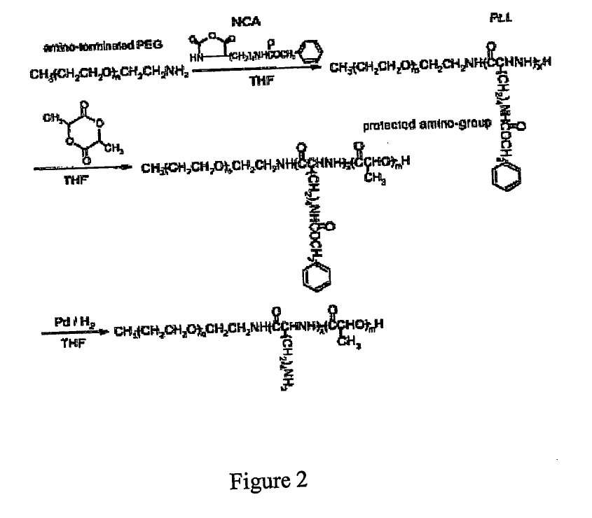

Amino-terminated PEG would initiate polymerization of NCA as described by Kricheldorf, which would yield PEG-b-N-CBZ-PLL. The end-amino group further initiates the polymerization of lactide resulting in ELB (“E”=hydrophilic block, “L”=charged block, and “B”=hydrophobic block). After deprotection of the N-CBZ group, the tri-block copolymer PEG-b-PLL-b-PLA could be obtained. This synthesis is shown in FIG. 2.

### EXAMPLE 3

### Tri-Arm Star Block Synthesis

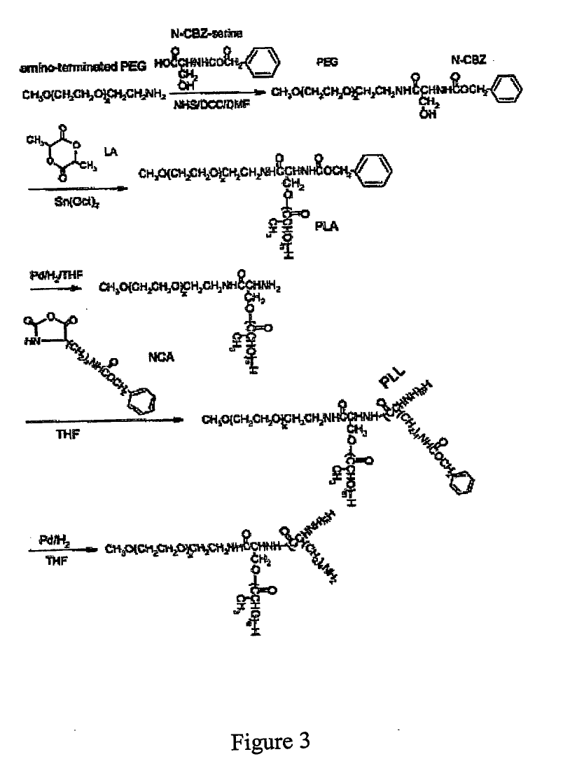

The first step is to modify the PEG end groups with N-CBZ serine so that PEG carries two different functional groups, one being the hydroxyl group and the other being the protected amino group. The second step is to use the hydroxyl group of the modified PEG to initiate the polymerization of lactide using Sn(Oct)₂ as catalyst in order to obtain the two-arm block copolymer of PEG and PLA with the remaining protected amino group. The third step is to deprotect the N-CBZ group of the two-arm block copolymer in order to activate the amino group. Finally, the protected polylysine is conjugated to the copolymer by the ring-opening polymerization of NCA with the amino group as an initiator. The star three-arm block copolymer is obtained by deprotecting the pendant N-CBZ group. This synthesis is shown in FIG. 3.

### EXAMPLE 4

### Transfection of DNA Material

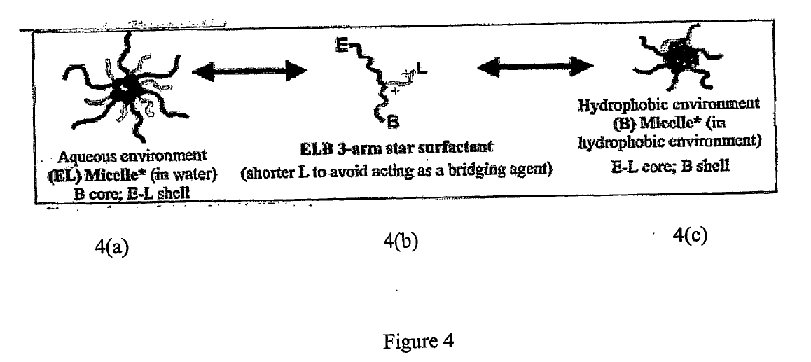

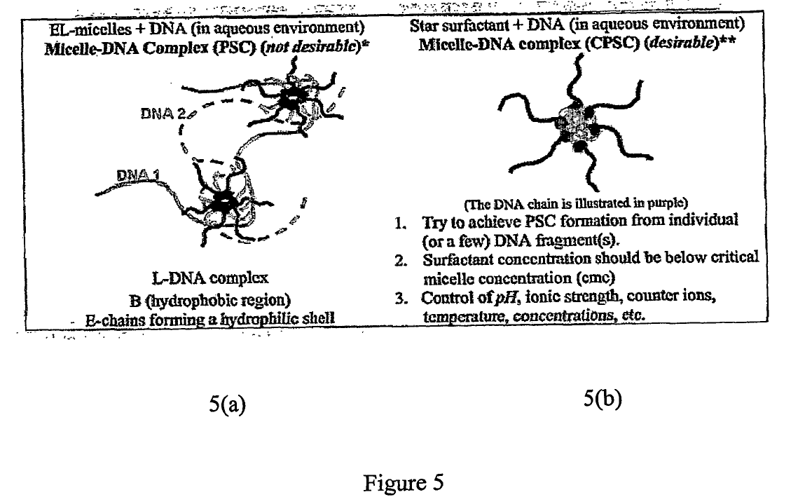

A schematic diagram of a micelle of 6 tri-block polymers FIG. 4(a) in equilibrium with an tri-arm star tri-block polymer FIG. 4(b) and the micelle formed in a hydrophobic environment is shown in FIG. 4(c). As shown in FIG. 5(a), the micelles are contacted with DNA strands in a hydrophilic environment and form a surfactant-DNA complex. This micelle-DNA complex in an aqueous environment is undersirable. Accordingly, preferably the DNA will first be condensed in a poor solvent, relatively hydrophobic solvent, and then the condensed DNA will be contacted with the surfactant, i.e. below its critical micelle concentration, in the poor, relatively hydrophobic, environment so that a strong DNA-charged block complex shell is formed on the surface of the condensed DNA. This supramolecular complex has both hydrophobic and hydrophilic blocks covalently bonded to the complex and is soluble in either the hydrophobic or the hydrophilic environment. Such a supramolecular complex can then become a soluble complex in the aqueous environement, as shown in FIG. 5(b). In order to avoid aggregation, it is permissible to neutralize amounts of both positive charges from the surfactant and negative charges from the polyeletrolyte in the CPSC so as to avoid further aggregation of the CPSC.

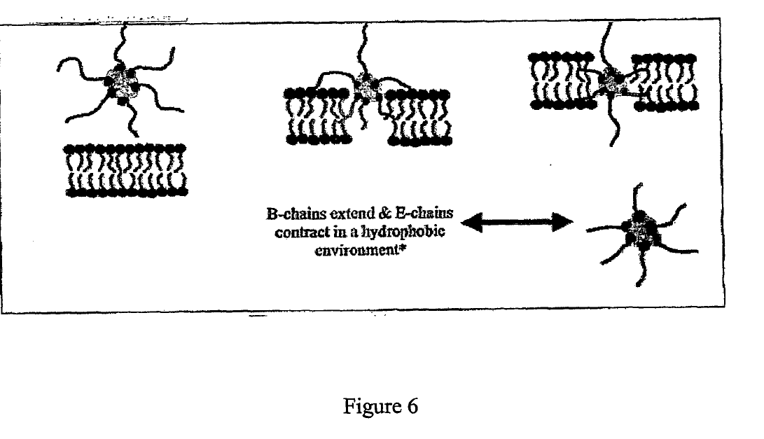

Transfection is schematically illustrated in FIG. 6. The duality in solubility of the CPSC should promote particle transmission through the cell membrane. In the hydrophobic region of the cell membrane, the B-chains should extend while the E-chains should collapse. The chain extensions and contractions will increase the transfection efficiency.

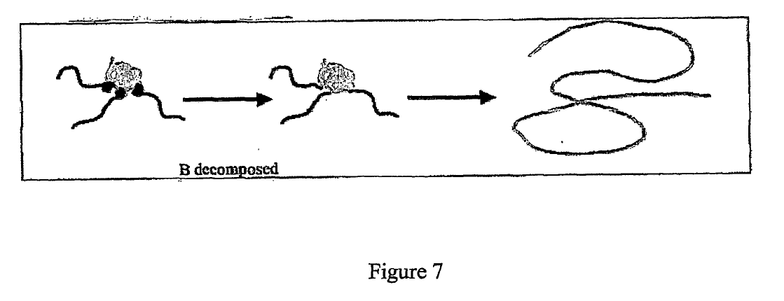

The disassembly of CPSC is shown in FIG. 7. Inside the cell, the hydrophilic E and hydrophobic B regions on the surface of the core should provide better protection for DNA from attack by nucleases. With biodegradation of the B-chains, the CPSC will destabilize. This process should provide easier access of the DNA by the nucleus. The positively charged L-chains are still covalently bound to the hydrophilic E-chains making these components less toxic and easier for discharge from the cell.

### EXAMPLE 5

### Synthesis of N2.N6-bis((phenylmethoxy)carbonyl)-L-lysine(2)

This synthesis is in accordance with Galbiati, B.; Ferrario, T.; Merli, V. WP 0110851(2001). A 250 mL three-necked flask was loaded consecutively with water (20 mL), 1,4-dioxane (20 mL) and L-lysine (2.1 g, 14.4 mmol). The mixture was stirred until complete dissolution. The pH was adjusted to about 10.5 by addition of 30% NaOH. Benzylchloroformate (5.2 g, 30.6 mmol) was added while maintaining the pH at about 10˜11 by adding at the same time 30% NaOH. At the end of the addition, the reaction was stirred at 20° C. for about 1 hour. The pH was adjusted to 5 with 37% HCl. Ethyl acetate (30 mL) was added and the pH was adjusted to 1 with 37% HCl. The mixture was stirred at room temperature for about 30 minutes, the organic layer was separated and the aqueous layer was extracted with ethyl acetate (20 mL). The combined organic layer was washed with brine (30 mL), and dried over Na₂SO₄. Then, the solvent was evaporated to yield a yellowish oil (6.0 g, 99%). The oil was pre-dried by azeotropic distillation with benzene.

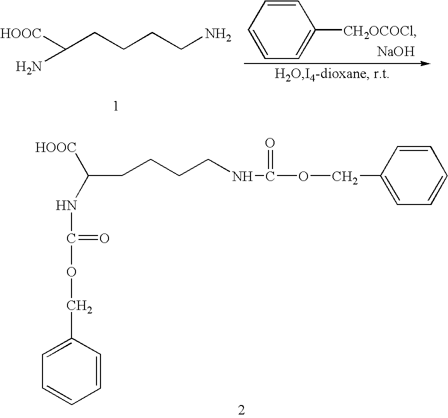

### EXAMPLE 6

### Synthesis of N5-((phenylmethoxy)carbonyl)-L-lysine, N-carboxyanhydride (NCA)

This synthesis was performed in accordance with Cannata, V.; Merli, V.; Sagwatti, S. EP 943621 (1999). Compound 2 (6.0 g, 14 mmol) was dissolved in dichloromethane (40 mL). To this solution, dimethyl formamide, DMF (1.5 mL) was added. The mixture was cooled to 0° C. Under stirring, thionyl chloride (2.32 g, 19 mmol) was added during 15 minutes. The reaction mixture was kept for one hour at 0° C., and then at 10° C. for a further 2 hours. After that it was evaporated under vacuum, and further dried in vacuum at 40-50° C. for 12 hours. A yellow-orange oil (4.5 g, 99%) was obtained. Compound NCA was obtained in this way at a purity of 90% as deteremined by NMR spectroscopy.

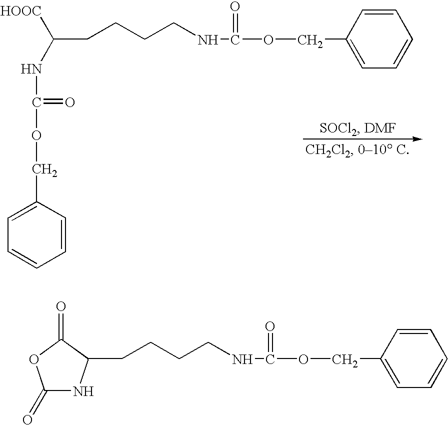

The tri-functional surfactant of the invention has a hydrophilic block (E), a charged block (L) and an additional biodegradable and flexible hydrophobic block (B). This third biodegradable hydrophobic (B) block can serve at least four useful functions. (1) It can modify the hydrophobic surface of the DNA-surfactant complex segments. (2) The supramolecular complex can be designed to be soluble not only in the aqueous environment but also in the hydrophobic environment. An increase in the compatibility with the interior of bilayer cell membranes should also promote the penetration of such complexes across the cell membrane. (3) The presence of a duality of hydrophobic and hydrophilic chains on the complex surface could increase the protection of genes in the supramolecular core. (4) The biodegradable block can be designed to destabilize the complex for eventual release of entrapped DNA chains.

The above description is illustrative and not limiting. Further modifications will be apparent to one of ordinary skill in the art in light of the disclosure and appended claims.

I claim:

1. A surfactant comprising a triblock copolymer including a hydrophilic block, a charged water-soluble block and a hydrophobic block.

2. A surfactant according to claim 1 wherein the charged water-soluble block is selected from the group consisting of a polyamino acid, chitin, chitosan, and polysaccharide.

3. A surfactant according to claim 2 wherein the polyamino acid is selected from the group consisting of poly(lysine), poly(arginine) and poly(ornithine).

4. A surfactant according to claim 1 wherein the hydrophobic block is selected from the group consisting of polylactide, polylactide-co-glycolide, poly(dioxanone), polyanhydride, sebacic acid, poly(ε-caprolactone), chitin, chitosan, poly(hydroxybutyrate) and poly(glycolide).

5. A surfactant according to claim 1 wherein the hydrophilic block is selected from the group consisting of polyethylene oxide, polyethylene glycol and polypropylene oxide.

6. A composition comprising a surfactant complex according to claim 1 and at least one selected from the group consisting of RNA and DNA.

7. A composition comprising a surfactant according to claim 1 and a pharmaceutical.

8. A method of delivering DNA to a tissue comprising forming a complex including a surfactant according to claim 1 and DNA, and contacting the tissue with the complex.
